(19) United States
(12) Patent Application Publication
(10) Pub. No.: US 2022/0401009 A1
(43) Pub. Date: Dec. 22, 2022

(54) SYSTEM, METHOD, AND HEAD MOUNTED DISPLAY FOR CONSUSSION ASSESSMENT

(71) Applicant: Virtual Reality Concussion Assessment Corporation, Raleigh, NC (US)

(72) Inventors: Shihching Yeh, Raleigh, NC (US); Hsiao-Kuang Wu, Raleigh, NC (US)

(73) Assignee: Virtual Reality Concussion Assessment Corporation, Raleigh, NC (US)

(21) Appl. No.: 17/353,793

(22) Filed: Jun. 21, 2021

(51) Int. Cl. A61B 5/00; A61B 5/378; A61B 5/16; A63F 13/26; A63F 13/213

(52) U.S. Cl. CPC A61B 5/4064; A61B 5/378; A61B 5/742; A61B 5/163; A61B 5/0006; A61B 5/0022; A63F 13/26; A63F 13/213; G06F 3/147

(57) ABSTRACT

A system for concussion assessment, comprises a head mounted display and a remote AI host. The head mounted display is disposed for displaying at least one test image to a user and obtain a plurality of physiological data from the user. The remote AI host communicates with the head mounted display through a first network connection, so as to obtain a concussion assessment result according to the plurality of physiological data.

### BACKGROUND OF THE INVENTION

### 1. Field of the Invention

The present disclosure relates to an assessment system, and in particular to a system for concussion assessment.

### 2. Description of the Related Art

The brain trauma are of mild traumatic brain injuries (mTBI) such as concussion, which is frequently-occurring on the people who take strenuous exercise or training such like basketball player, football player, boxer, brawler, soldier, etc. There are over 1 million mTBI cases in USA each year.

In many instances, concussions produce a disturbance of brain function rather than structural injury. Accordingly, persons affected by mTBI often demonstrate no immediate post-trauma abnormalities. Concussive trauma may nevertheless result in mechanical injury to the axonal component that can result in acute to long-term damage and axonal degeneration. Conventional models for mTBI are complex and require significant contextual and subject-specific information. These conventional models are difficult to deploy for use in the field, whether for occupational health hazards including trauma-inducing events occurring during sporting events or combat.

However, common diagnostic brain imaging device, such as CT, MRI, PET scan, and fMRI cannot detect concussion effectively. Therefore, the golden treatment period of concussion is mistaken because the prior art cannot immediately and preciously diagnosis concussion.

### BRIEF SUMMARY OF THE INVENTION

The present disclosure provides a system, which is able to assess concussion.

The present disclosure further provides a head mounted display, which is able to obtain some personal information for concussion assessment.

The present disclosure also provides a method for concussion assessment.

The present disclosure provides a system for concussion assessment, comprises a head mounted display and a remote AI host. The head mounted display is disposed for displaying at least one test image to a user and obtaining a plurality of physiological data from the user. The remote AI host communicates with the head mounted display through a first network connection, so as to obtain a concussion assessment result according to the plurality of physiological data.

The system provided by the present disclosure further comprises a portable device. The portable device communicates with the head mounted display through a second network connection, as well as the remote AI host communicates with the portable device through the first network connection. Therefore, the portable device is able to transmit the plurality of physiological data to the remote AI host from the head mounted display.

In some embodiments, the first network connection is WIFI, LAN or mobile network, and the second network connection is Bluetooth, ZigBee, infrared, ANT, WIFI, or LAN.

Furthermore, the present disclosure provides a head mounted display, which is suitable for concussion assessment, comprising a controller, a display module, and a detection module. The display module is coupled to the controller, such that the controller makes the display module displaying at least one test image. The detection module is disposed for obtaining a plurality of physiological data from a user upon displaying the at test one test image.

The display module in the head mounted display comprises a display panel and a lens set. The display panel is disposed for displaying the at least one test image. The lens set is disposed for projecting the at least one test image to the user's eyes.

In some embodiments, the head mounted display provided by the present disclosure further comprises a case and a portable device. The portable device has a screen for displaying the at least one test image, and the portable device is suit for installing on the case. Wherein the display module has a lens set for projecting the at least one test image to the user's eyes.

In some embodiments, the detection module comprises an eyes tracking detector and a brain waves detector. The eyes tracking detector detects the movement of the user's eyes and outputs a first detection data to the controller. The brain waves detector detects the user's brain waves and outputs a second detection data to the controller. Wherein the first detection data and the second detection data are involved in the physiological data.

In some embodiments, the system provided by the present disclosure further comprises a communication module, which is coupled to the controller, so as to transmit the plurality of physiological data on a network connection.

Furthermore, the present disclosure provides a method for concussion assessment comprising at least of the following steps. Displaying at least one test image on a head mounted display. Obtaining a plurality of physiological data from a user through the head mounted display upon displaying the at least one test image. Transmitting the plurality of physiological data to a remote AI host. Performing an algorithm on the remote AI host to fuse the plurality of physiological data for obtaining a concussion assessment result according to the plurality of physiological data.

In some embodiments, the method provided by the present disclosure comprises the step of sending the concussion assessment result to a portable device.

In some embodiments, the step of displaying the least one test image is displaying at least one 3D VR game animation or 3D VR interact sense game animation.

Compared with the prior art, the present disclosure is portable, such that it can be used in many situations. In addition, the head mounted display is able to communicates with the remote AI host, as well as the remote AI host is able to communicates with a portable, such like mobile phone, such that the care-giver can obtain the concussion immediately by the mobile phone.

### BRIEF DESCRIPTION OF THE DRAWINGS

FIG. 1 is a schematic diagram of the system for concussion assessment according to a first embodiment of the present disclosure.

FIG. 2 is a schematic diagram of the HMD.

FIG. 3 is a block diagram of the HMD according to an embodiment of the present disclosure.

FIG. 4 is a schematic diagram of the system for concussion assessment according to a second embodiment of the present disclosure.

FIG. 5 is a flow chart of a method for concussion assessment according to an embodiment of the present disclosure.

FIG. 6 shows VR game animations for brain waves detection according to an embodiment of the present disclosure.

FIG. 7 shows VR game animations for eyes tracking detection according to an embodiment of the present disclosure.

FIG. 8 shows the images and features of brain waves according to an embodiment of the present disclosure.

FIG. 9 shows the images and features of the movement of user's eyes according to an embodiment of the present disclosure.

### DETAILED DESCRIPTION OF THE INVENTION

To facilitate understanding of the object, characteristics and effects of this present disclosure, embodiments together with the attached drawings for the detailed description of the present disclosure are provided.

In the following paragraphs, A “couples to” B may mean A directly or in-directly connecting to B, such like A connects to B by active or passive component. Furthermore, the meaning of “couple” may involve the analog electric signal or digital data exchanging through wire or wireless connection.

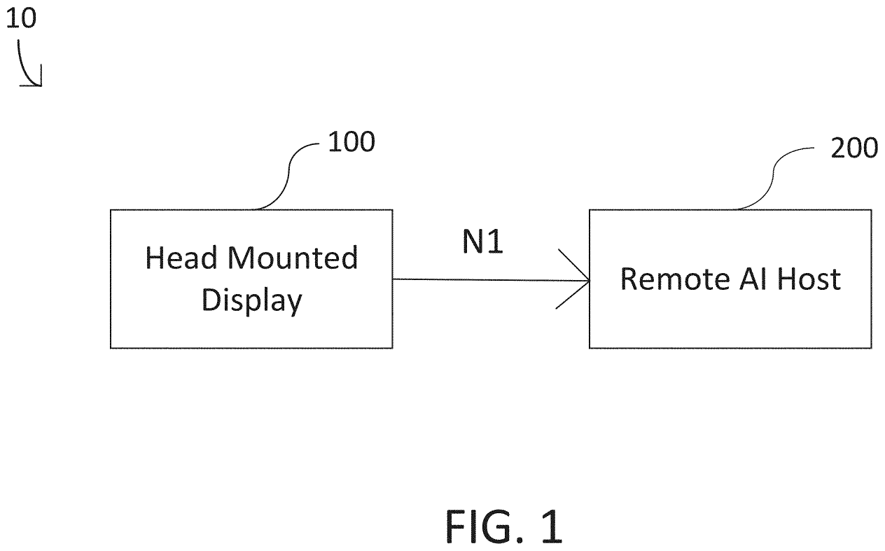

FIG. 1 is a schematic diagram of the system for concussion assessment according to a first embodiment of the present disclosure. Please referring to FIG. 1, the system 10 for concussion assessment comprises a head mounted display 100 (refer to “HMD 100” hereinafter) and a remote AI host. In some embodiments, the remote AI host communicates with the HMD 10 by a first network connection N1.

In some embodiments, the HMD 10 is able to display at least one test image and obtain a plurality of physiological data from a user when the user is watching the test image. In exemplary, the HMD 10 may be implemented by a virtual reality (VR) HMD. Through the immersive experience by VR HMD, the HMD 10 may obtain correct physiological data. In those embodiments, the test image may be a VR game animation.

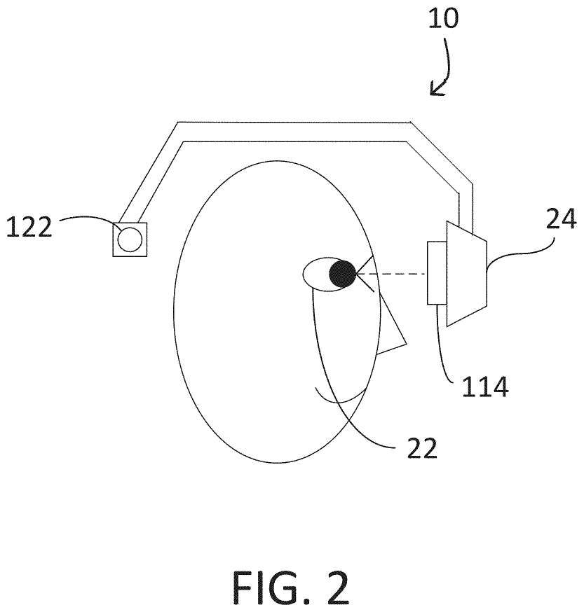

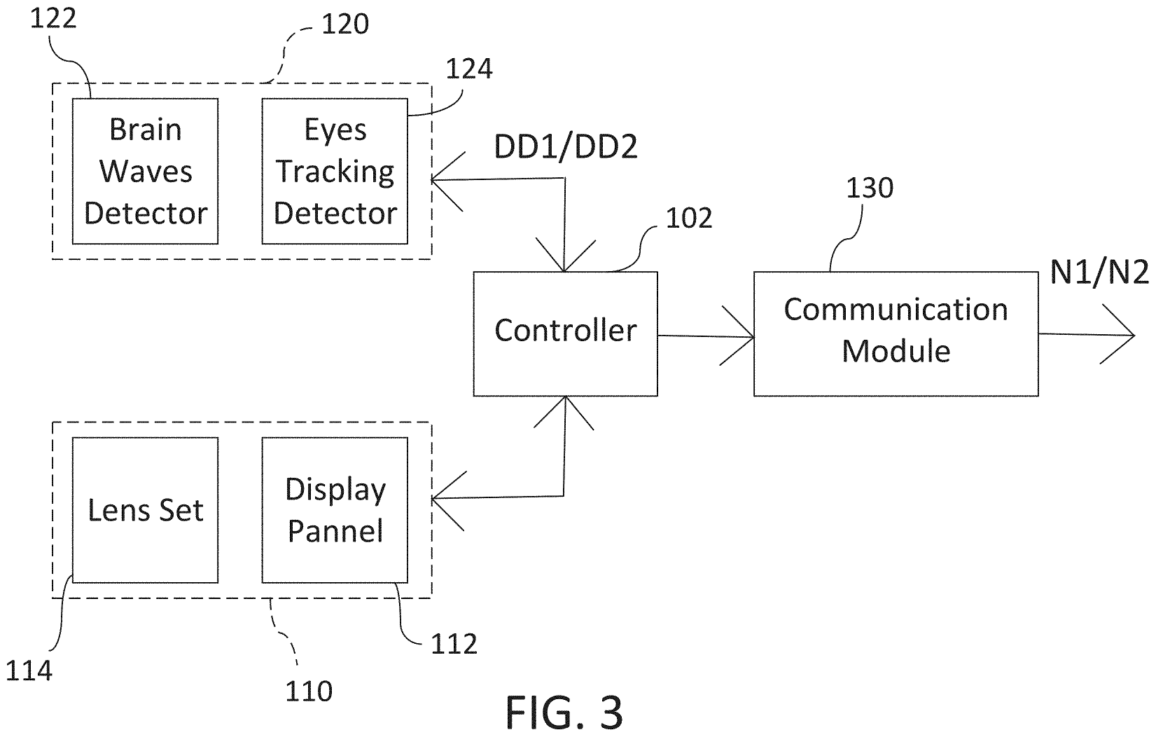

FIG. 1 is a schematic diagram of the system for concussion assessment according to a first embodiment of the present disclosure; and FIG. 3 is the block diagram of the HMD according to an embodiment of the present disclosure. Please refer to FIG. 2 and FIG. 3. HMD 10 has a controller 102, a display module 110 and a detector module 120. Controller 102 may be coupled to the display module 110, so as to make display module 110 displaying the test image. In some embodiments, the display module 110 may comprises a display panel 112 and a lens set 114. The panel 112, which is able to display the test image, may be implemented by LCD, OLED, or micro-LED panel. The lens set 114 may be disposed between the human's eyes 22 and display panel 112. Accordingly, the lens set 114 could project the test image to the user's eyes 22.

In other embodiments, the display panel 112 may be replaced by a portable device (not shown), such like mobile phone. The portable device has a screen, which is in charge of displaying the test image. In those embodiments, the portable device may be installed on the case 24 of the HMD 10, and the screen of the portable device may be toward to the lens set 114. Such that, the lens set 114 is able to project the test image shown by the portable device to the user's eyes 22.

Referring to FIG. 3, detector module 120 comprises, for example, a brain waves detector 122 and an eyes tracking detector 124. When the display module 110 displays the test image, the controller 102 may enable detector module 120 to obtain a plurality of the physiological data. In some embodiments, the detector module 12 comprises brain waves detector 122 and eyes tracking detector 124. In those embodiments, the brain waves detector 122 may be an electroencephalogram electrode and disposed on back side of the HMD 10, such that the brain waves detector 122 can contact the user's head when he/she wear the HMD 10. Accordingly, the controller 102 is able to enable the brain waves detector 122 to detect the variation of the user's brain waves when he/she watches the test image, and then, the brain waves detector 122 outputs a first detection data DD1 to the controller 102. Meanwhile, the eyes tracking detector 124 is able to detect the movement of user's eyes and output a second detector data DD2. Wherein, both of the first detection data DD1 and the second detection data DD2 are involved in aforementioned physiological data.

In an exemplary, the HMD10 further has a communication module 130. When detection module 120 obtain the physiological data, which are sent to the controller 102, and then, the controller 102 transmits the physiological data to the communication module 130. Meanwhile, the communication module 130 is able to communicate with the remote AI module host 200 by a first network connection N1, and transmit the physiological data to the remote AI host 120 to perform the concussion assessment. In some embodiments, the first network connection N1 is a wireless connection, such like WIFI, LAN, mobile network

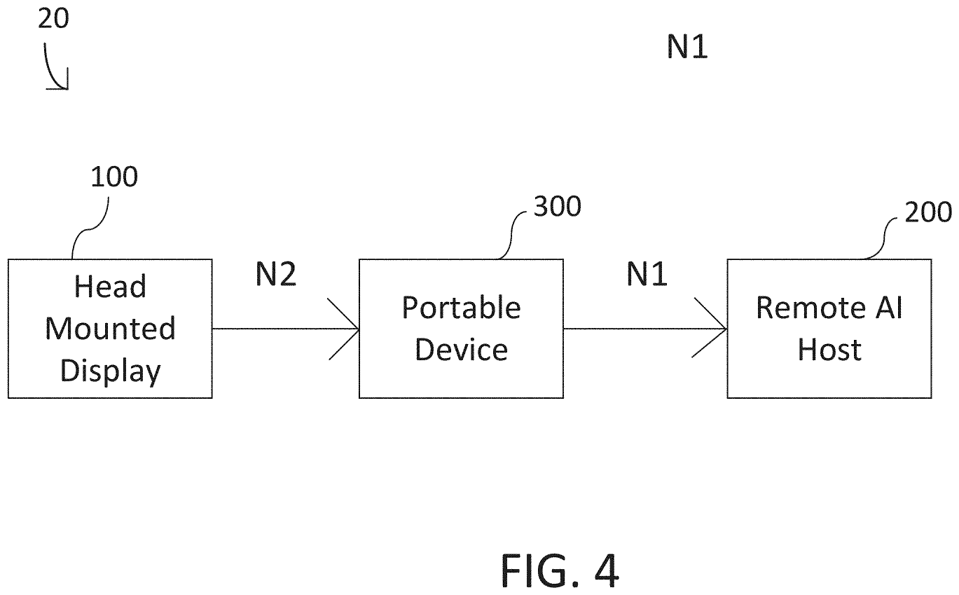

When the remote AI host 120 received the physiological data, it would obtain some images and features from the physiological data. Please referring to the FIG. 4, in accordance with the first detector data DD1, the remote AI host 120 is able to obtain the brain waves images, as well as their corresponding features. Furthermore, the remote AI host 120 is able to obtain the eyes tracking images and features by the specific algorithm according to the second detection data DD2. When those images and features are obtained, the remote AI host, in some embodiments, would enable a machine learning process to deal with those images and features. Therefore, the remote AI host 120 is able to fuse those images and features for obtaining a concussion assessment result. A care-giver is able to assess whether the user has the tendency of concussion by forementioned assessment result. The images and features will be described in detail in below.

Please referring to FIG. 3, in other embodiments, the controller 102 has an embedded system, MCU (Not Shown) or sub-system, which is able to obtain the forementioned images and features. Then, the controller 102 would transmit the images and features to the communicate module 130 and send those to the remote AI host 200, so as to fuse those images and feature to obtain the concussion assessment result.

FIG. 4 is a schematic diagram of the system for concussion assessment according to a second embodiment of the present disclosure. Please referring to FIG. 4, the system 20 for concussion assessment in the second embodiment is similar with the system 10. Furthermore, the system 20 comprises a portable device 300. In some embodiments, the portable device 300, for example, is a mobile phone, a pocket PC, a tablet PC, a notebook PC, a hand-held industrial PC, or a hand-held nursery PC.

Please refer to FIG. 3 and FIG. 4, the HMD 10 is able to communicate with the portable device 300 through a second network connection N2. In some embodiments, the second network connection N2 is Bluetooth, ZigBee, infrared, ANT, WIFI, or LAN. In contrary, the portable device 300 is able to communicate with the remote AI host 200 through the first network N1. In some embodiments, the first network connection N2 is a wireless network, such like WIFI, LAN, or a mobile network. Accordingly, the HMD 10 is able to transmit the physiological data to the portable device 300 by the second network connection N2, and then, the portable device 300 transmits the physiological data to the remote AI host 200 through the first network connection N1. When the remote AI host 200 obtains the concussion assessment result, the concussion assessment result could be transmitted to the portable deice 300. Therefore, the care-giver is able to know the concussion assessment result through the portable device 300.

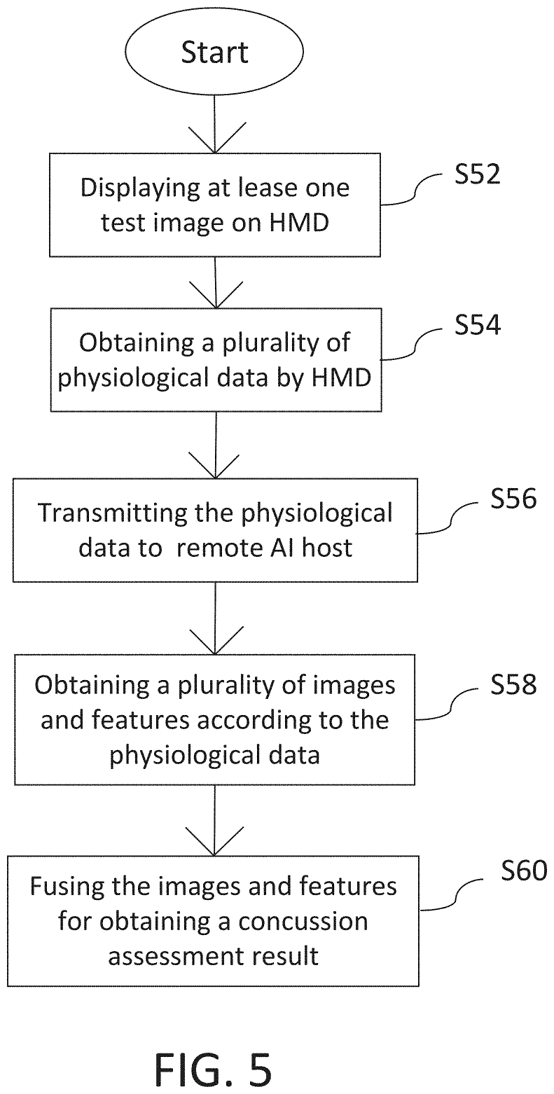

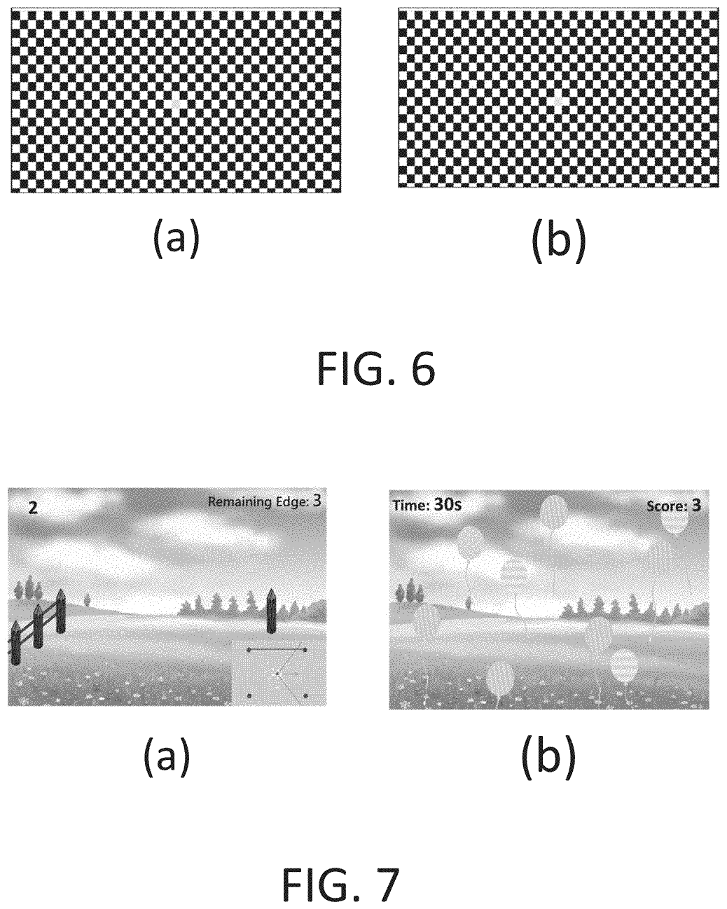

FIG. 5 is a flow chart of a method for concussion assessment according to an embodiment of the present disclosure. Please referring to FIG. 5, in step S52, at least one test image is displayed on an HMD. In exemplary, the test image is able to be implemented by 3D VR game animation or 3D VR interact game animation, such like FIG. 6 and FIG. 7. In those embodiments, FIGS. 6(a) and 6(b) shows the 3D VR animation for brain waves detection. When displaying the amination in FIG. 6, the images in FIGS. 6(a) and 6(b) would be switched continually. Additionally, the animations in FIG. 7 may be for eyes tracking detection. In some embodiments, the 3D VR interact sense animation in FIG. 7(a) is for eyes tracking saccades detection, and the animation in FIG. 7(b) is for eyes tracking fixations. In detail, when displaying the 3D VR interact sense animation show as FIG. 7 on the HMD, the user is able to control the stakes or balloons by moving his or her eyes, so as to obtain corresponding scores. For understanding, although FIG. 6 and FIG. 7 provide some examples for the test images, the present disclosure is not limited thereto.

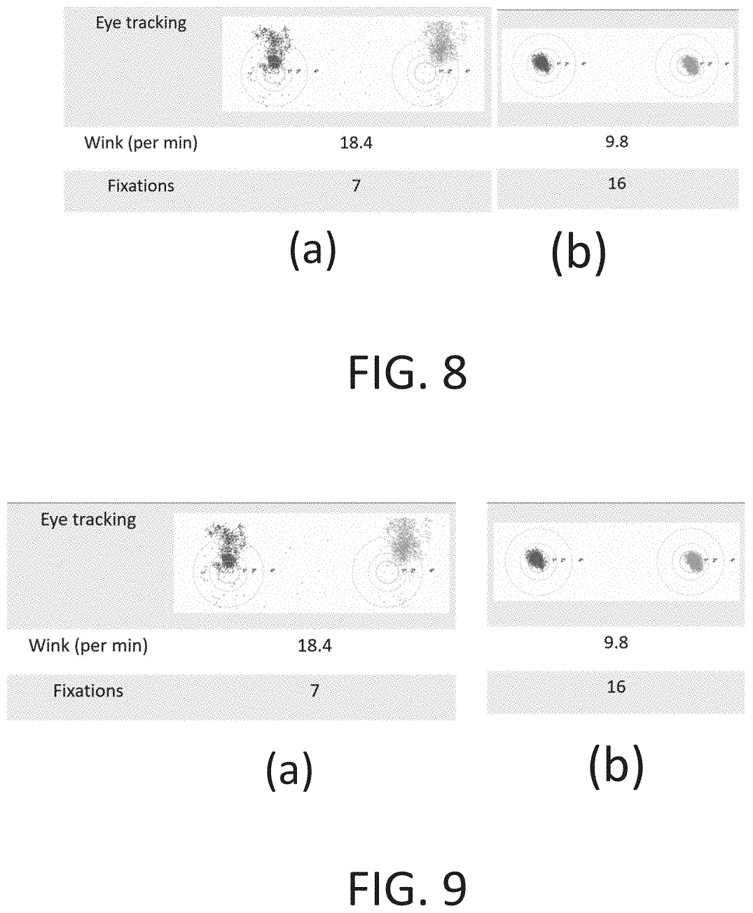

In step S54, a plurality of physiological data can be obtained through the HMD from a user upon displaying the test images. For example, when the HMD displays the 3D VR game animation as shown in FIG. 6, it is able to detect the user's brain waves as the first detection data. Similarly, when the 3D VR interact sense game animations shown in FIG. 7 is displayed, the HMD is able to count the forementioned scores and corresponding spend time, which are the second detection data. Wherein, the first and second detection data are involved in the physiological data. Then, the physiological data is transmitted to a remote AI host, such as the step S56. Going to the step S58, the remote AI host is able to obtain a plurality of images and features according to the physiological data, such as shown in FIG. 8, FIG. 9 and FIG. 10. FIG. 8 shows the images and features about user's brain waves. Wherein, the features are the SNR values. FIG. 8(a) and FIG. 8(c) shows normal data. In contrary, the image and feature in FIG. 8(b) represent that the user has high possibility to suffer a concussion.

FIG. 9 shows the images and features for eye fixations indication. Wherein, the feature is the frequency of winking per minute. The eye fixations indication is more less, the possibility of suffering the concussion is higher. Such that, the image and feature shown in FIG. 9(b) from a user who may be normal, and the image and feature shown in FIG. 9(a) represent that the user has high possibility of unnormal. FIG. 10 shows the images and features for eye saccades indication. Differently, when the value of eye saccades is higher, the possibility of suffering the concussion is higher. Accordingly, the image and feature in FIG. 10(b) mean that the user may be normal, and those shown in FIG. 10(a) tell us that the user may be unnormal. In some embodiments, when the remote AI host obtain those images and features, it would enable a machine learning process or deep learning process to deal with those images and features for training an algorithm installed on the remote AI hose. Accordingly, in the step S60, The remote AI host would fusing, for example by the algorithm, the physiological data for obtaining a concussion assessment result.

In some embodiments, when the concussion result is obtained, it would be transmitted to a portable device through a network connection.

Alternately, in other embodiments, the images and features in the step S58 may be obtained by HMD. In those embodiments, the HMD would transmit those images and features to the remote AI host through a network connection or through a portable device.

In summary, the present disclosure has some characteristics as following.

(1) The present disclosure adopts HMD to detect user's physiological and upload to the remote AI host directly or through portable device, such like mobile phone or tablet PC. Such that, the present disclosure is easy to carry. Therefore, the care-giver can assess whether a user has tendency of concussion immediately and conveniently.

(2) The present disclosure obtains the assessment result by machine learning for the images and features of user's brain waves data and eyes motion data and fusing those. Accordingly, the assessment result obtained by the present disclosure is more precious and reliable.

(3) In the present disclosure, when the remote AI host obtain the images and features, it would enable a machine learning or deep learning process to deal with those images and features for training its algorithm. Therefore, the algorithm has higher accuracy.

(4) Since the present disclosure deploys HMD to detect user's physiological data, it is easy to operate for everyone. Moreover, the assessment result can be displayed on a portable device. Therefore, it is easy to operate for the care-giver even who doesn't have related skill or knowledge.

While the present disclosure has been described by means of specific embodiments, numerous modifications and variations could be made thereto by those skilled in the art without departing from the scope and spirit of the present disclosure set forth in the claims.

What is claimed is:

1. A head mounted display, suitable for concussion assessment, comprising:
a controller;
a display module, coupled to the controller, such that the controller makes the display module displaying at least one test image; and
a detection module, disposed for obtaining a plurality of physiological data from a user.

2. The head mounted display of claim 1, wherein the display module comprises:
a display panel, disposed for displaying the least one test image; and
a lens set, disposed for projecting the least one test image to the user's eyes.

3. The head mounted display of claim 1, further comprising:
a case; and
a portable device, has a screen for displaying the least one test image, and the portable device is installed on the case,
wherein the display module has a lens set for projecting the least one test image to the user's eyes.

4. The head mounted display of claim 1, wherein the detection module comprises:
an eyes tracking detector, detects the movement of the user's eyes, and outputs a first detection data to the controller; and
a brain waves detector, detects the user's brain waves, and outputs a second detection data to the controller,
wherein the first detection data and the second detection data are involved in the physiological data.

5. The head mounted display of claim 4, wherein the brain waves detector is an electroencephalogram electrode, coupled to the controller and disposed on back side of the head mounted display.

6. The head mounted display of claim 3, further comprises:
a communication module, coupled to the controller, so as to transmit the plurality of physiological data to a network connection.

7. The head mounted display of claim 5, wherein the network connection is Bluetooth, ZigBee, infrared, ANT, wireless network, WIFI, LAN or mobile network.

8. A method for concussion assessment, comprising at least of the following steps:
displaying at least one test image on a head mounted display;
obtaining a plurality of physiological data from a user through the head mounted display upon displaying the least one test image;
transmitting the plurality of physiological data to a remote AI host; and
performing an algorithm on the remote AI host to fuse the plurality of physiological data for obtaining a concussion assessment result.

9. The method of claim 8, further comprising the step:
sending the concussion assessment result to a portable device.

10. The method of claim 9, wherein the step of displaying the least one test image is displaying at least one 3D VR game animation or 3D VR interact sense game animation.

11. The method of claim 10, wherein the step of obtaining the plurality of physiological data comprises:
detecting the movement of the user's eyes to obtain an eye tracking saccades data and an eye tracking fixations data upon displaying a 3D VR interact sense game animation on the head mounted display; and
detecting the user's brain waves data upon displaying a 3D VR game animation on the head mounted display.

12. The method of claim 8, further comprising the following steps:
transmitting the plurality of physiological data to a portable device through a first network connection; and
transmitting the plurality of physiological data to the remote AI host from the portable device through a second network connection.

13. The method of claim 12, wherein the first network connection is Bluetooth, ZigBee, infrared, ANT, WIFI, or LAN.

14. The method of claim 12, wherein the second network connection is WIFI, LAN, or a mobile network.

15. A system for concussion assessment, comprising:
a head mounted display, disposed for displaying at least one test image to a user and obtain a plurality of physiological data from the user; and
a remote AI host, communicates with the head mounted display through a first network connection, so as to fuse the plurality of the physiological data for obtaining a concussion assessment result.

16. The system of claim 15, further comprising:
a portable device, communicates with the head mounted display through a second network connection and the remote AI host through the first network connection.

17. The head mounted display of claim 16, wherein the first network connection is WIFI, LAN or a mobile network.

18. The head mounted display of claim 16, wherein the second network connection is Bluetooth, ZigBee, infrared, ANT, WIFI, or LAN.

19. The system of claim 16, wherein the portable device is a mobile phone, a pocket PC, a tablet PC, a notebook PC, a hand-held industrial PC, or a hand-held nursery PC.

20. The system of claim 15, wherein the head mounted display is VR head mounted display.
